(19) United States
(12) Patent Application Publication
(10) Pub. No.: US 2015/0320814 A1
(43) Pub. Date: Nov. 12, 2015

(54) NUTRACEUTICAL FORMULATION FOR TREATMENT OF ANXIETY AND DEPRESSION

(71) Applicant: Creative Medical Health Inc., Phoenix, AZ (US)

(72) Inventors: Amit Patel, Salt Lake City, UT (US); Timothy Warbington, Phoenix, AZ (US); Annette Marleau, San Diego, CA (US)

(21) Appl. No.: 14/707,992

(22) Filed: May 8, 2015

Related U.S. Application Data
(60) Provisional application No. 61/992,149, filed on May 12, 2014.

(51) Int. Cl. A61K 36/185; A61K 31/455; A61K 36/81; A61K 31/198; A61K 36/38; A61K 31/197; A61K 36/28; A61K 33/06; A61K 36/539

(52) U.S. Cl. CPC A61K 36/185; A61K 33/06; A61K 31/455; A61K 36/81; A61K 36/539; A61K 36/38; A61K 31/197; A61K 36/28; A61K 31/198

(57) ABSTRACT

Disclosed are compositions of matter useful for treating anxiety and depression. In one embodiment a nutraceutical composition is administered to a patient in need of said composition comprising of the following combination of ingredients: niacin, magnesium, ashwagandha, passionflower, skullcap, St. John's Wort, gamma aminobutryic acid, chamomile, and L-theanine

### CROSS-REFERENCE TO RELATED APPLICATIONS

This application claims priority to Provisional Application Ser. No. 61/992,149, filed May 12, 2014, and entitled “Nutraceutical Formulation for Treatment of Anxiety and Depression” which is hereby expressly incorporated by reference in its entirety.

### FIELD OF THE INVENTION

The present technology pertains to the field of naturopathic medicine for treating depression and anxiety. Specifically, the invention relates to the use of natural substances for management of anxiety and depression. More specifically, the invention relates to the use of combinations of niacin, magnesium, ashwagandha, passion flower, skullcap, St. John's Wort, gamma aminobutryic acid, chamomile, and L-theanine

### BACKGROUND

Anxiety disorders and depression represent a spectrum of disorders that cause significant psychological distress, interfering with an individual's quality of life, productivity and overall health status. While these disorders can be precipitated or exacerbated by specific environmental stressors, they each also have an explicit biological basis in the nervous system that will be reviewed briefly here as it pertains to the present invention.

Pharmaceuticals that have been developed to address these disorders include the benzodiazepines, a broad class of drugs used to treat anxiety, insomnia, seizures, depression, panic attacks, alcohol withdrawal, and other conditions. These drugs act by stimulating GABA(A) Benzodiazepine receptors. GABA, which can be considered the brain's “natural valium” is the primary inhibitory neurotransmitter in the central nervous system. By acting as GABA agonists, benzodiazepines stimulate these receptors to become more sensitive to GABA. In turn, this so-called “GABAergic activity” serves to and reduce excitability in the brain whereas even a mild attenuation of GABA activity can lead to arousal, anxiety, insomnia and restlessness [reviewed in [1]]. Examples of benzodiazepines include diazepam (Valium) and alprazolam (Xanax).

Other drugs for treating anxiety and/or depressive disorders include tricyclic antidepressants, monoamine oxidase inhibitors, selective serotonin reuptake inhibitors, and serotonin and norepinephrine reuptake inhibitors. Thus, while GABA is a major neurotransmitter pathway in the brain, drugs and/or alternative remedies for anxiety and depression can also have beneficial effects by changing the availability of other neurotransmitters in the brain, including serotonin, norepinephrine, and dopamine.

Since pharmaceuticals are not without side effects, and the impact of herbal remedies, vitamins and minerals on cognitive function have been recognized for centuries, the use of nutraceuticals for managing anxiety and depression represent attractive alternatives. Embodiments of the present invention encompass compositions of matter and methods for administering a nutraceutical formulation to treat anxiety and depression.

### SUMMARY OF THE INVENTION

Embodiments herein relate to a composition for preventing and managing depression and anxiety and their effects in a mammal comprising: niacin, magnesium, ashwagandha, passion flower, skullcap, St. John's Wort, gamma aminobutryic acid, chamomile, and L-theanine in an amount sufficient to effect stress and anxiety in said mammal.

Further embodiments relate to a method for reducing depression and anxiety in a mammal, said method comprising: identifying a mammal with stress and anxiety; administering a composition of naturally occurring substances comprising niacin, magnesium, ashwagandha, passion flower, skullcap, St. John's Wort, gamma aminobutryic acid, chamomile, and L-theanine in an amount sufficient to effect stress and anxiety in said mammal.

### DETAILED DESCRIPTION

Embodiments of the present invention are described below. It is, however, expressly noted that the present invention is not limited to these embodiments, but rather the intention is that modifications that are apparent to the person skilled in the art and equivalents thereof are also included. This invention teaches compositions of natural products for the management of anxiety and depression in a mammal. One of skill in the art utilizes said formulation as a monotherapy or in conjunction with treatment(s) known to address anxiety and depression. The nutraceutical formulation comprises the following ingredients: niacin, magnesium, ashwagandha, passion flower, skullcap, St. John's Wort, gamma aminobutryic acid, chamomile, and L-theanine.

This invention teaches compositions of natural products for the management of anxiety and depression in a mammal. One of skill in the art utilizes said formulation as a monotherapy or in conjunction with treatment(s) known to address anxiety and depression. The nutraceutical formulation comprises the following ingredients: niacin, magnesium, ashwagandha, passion flower, skullcap, St. John's Wort, gamma aminobutryic acid, chamomile, and L-theanine

The therapeutic properties of the various components of the composition have been previously described; however, utilization of these compositions in combination for managing depression and anxiety, have not been reported. In the current invention, therapeutic complications are associated with additive/synergistic effects of the named ingredients to manage depression and anxiety in mammals.

In one embodiment, niacin is administered to a mammal along with a nutraceutical formulation comprising ashwagandha, magnesium, passion flower, skullcap, St. John's Wort, gamma aminobutryic acid, chamomile, and L-theanine Niacin, also known as vitamin B3 and nicotinic acid, is a water-soluble B-complex vitamin that has numerous functions in the body; for example, cholesterol metabolism, production of steroid hormones in the body, and DNA repair, and in supporting healthy circulation and cognitive functions. Niacin can be acquired through dietary sources with rich food sources being meats, fish and prawns, peanuts, mushrooms, seaweed, potatoes, and peas.

It has been suggested that niacin supplementation increases the availability of the mood enhancing neurotransmitter serotonin through an indirect mechanism. Both niacin and serotonin are biosynthesized from the amino acid L-tryptophan in the liver. On a molar basis, approximately 60 mg of dietary tryptophan is equivalent to 1 mg of niacin [2]. Dietary supplementation with niacin is therefore thought to facilitate greater utilization of L-tryptophan toward generating serotonin. Depression is associated with low levels of serotonin, as supported by the beneficial effects of a class of drugs known as selective serotonin uptake inhibitors such as Prozac. Niacin is a natural alternative to prescription drugs for treatment of anxiety and depression. In its most severe form, niacin deficiency causes pellagra, a disease that resembles schizophrenia that is marked by impaired by disturbances in psychosensory, psychomotor, and emotional functions [3]. Depression that is associated with pellagra is attributed to deficiency of serotonin in the brain, resulting from decreased availability of tryptophan. In an early report (1953), 14 of 15 patients with clinical depression treated with nicotinic acid showed significant improvements in mood [4]. The authors suggested that these effects might be owing to its actions as a vasodilator, providing improved cerebral circulation.

In another embodiment, ashwagandha is administered to a mammal along with said composition of ingredients comprising niacin, magnesium, ashwagandha, passionflower, skullcap, St. John's Wort, gamma aminobutryic acid, chamomile, and L-theanine Ashwagandha (Withania somnifera) is an exotic Indian herb has been used in India for thousands of years as a rejuvenating tonifier and to support vitality. It is known in the art of naturopathic medicine that ashwagandha is considered an “adaptogen”—a substance that helps the body adapt to physiological and psychological stress, having a balancing effect on the body's systems and regulating the stress hormone-producing glands. Ashwagandha been demonstrated to be as effective as some tranquilizers and antidepressant drugs against anxiety and depression [5].

The concentrations of ashwagandha needed for anxiolytic effects in patients in need of said intervention are known in the art. In a published study of 64 healthy subjects with a history of chronic stress that were randomized to take extract of ashwaghanda root (300 mg capsules taken twice daily) or placebo, the group that took ashwaganda scored significantly lower on anxiety scores after two months [6]. Serum cortisol levels, serving as physiologic indicators of stress levels, were also significantly reduced in the ashwagandha-treated patients. In one embodiment of the present invention, a method of alleviating anxiety and/or depression in a mammal involves administering a concentration of ashwagandha between 100-500 mg along with niacin, magnesium, skullcap, St. John's wort, gamma aminobutyric acid, chamomile, and L-theanine

In another embodiment, passionflower is administered to a mammal along with said composition of ingredients comprising niacin, ashwagandha, magnesium, skullcap, St. John's wort, gamma aminobutryic acid, chamomile, and L-theanine. This plant (Passiflora incarnate L.) is a traditional remedy for treating for anxiety, insomnia and hysteria.

In two clinical trials, Passiflora extracts demonstrated anxiolytic effects that were similar to those of benzodiazepines, as well as having sedative effects [7, 8]. From a mechanistic standpoint, there is evidence that Passiflora extract stimulates gamma aminobutyric acid (GABA) in the brain, thereby having similar effects to the prescription drug diazepam, a tranquilizer [9].

In another embodiment, magnesium is administered to a mammal along with said composition of ingredients comprising niacin, passionflower, ashwagandha, skullcap, St. John's wort, gamma aminobutryic acid, chamomile, and L-theanine Magnesium is an essential dietary mineral that has been used as an oral supplement for combating depression [10]. It is known in the art that magnesium deficiency causes neuropathologies, possibly due to its role in neuronal signal transduction. A published report summarizing case histories revealed that magnesium deficiency is accompanied by a spectrum of cognitive defects, including anxiety, insomnia, irritability, postpartum depression, and short tem memory loss [11]. Rapid recovery from major depression can be afforded by supplementation of patients with 125-300 mg of magnesium with each meal and at bedtime. In one embodiment of the present invention, magnesium is administered to an individual suffering from depression and/or anxiety at concentrations between 50-1000 mg per serving.

In another embodiment, skullcap is administered to a mammal along with said composition of ingredients comprising niacin, ashwaganda, magnesium, passionflower, St. John's Wort, gamma aminobutryic acid, chamomile, and L-theanine Skullcap (Scutellaria sp.) is an herb commonly used in traditional Chinese medicine to calm tension, ease spasms and promote restfulness, known in the art as a “nerve tonic”, which was commonly used long before pharmaceutical tranquilizers were available.

Skullcap is available as powder, fluid extract and tea, and is rich in flavonoids such as wogonin, baicalin, and baicalein, to which its biological activities have been attributed. In a published double blind, placebo-controlled study of healthy subjects, skullcap demonstrated anxiolytic effects [12]. Mechanistically, anti-oxidant actions of flavonoids in skullcap are implicated in its beneficial effects against neurodegenerative and neuropsychiatric disorders [13]. As known in the art, these anti-oxidant compounds scavenge and neutralize free radicals that are responsible for cellular damage. Specifically, the antioxidants baicalin and baicalein isolated from skullcap have been identified as agonists of GABA receptors, thereby engaging a major inhibitory pathway in the CNS that reduces anxiety [14].

In another embodiment, St. John's wort is administered to a mammal along with said composition of ingredients comprising niacin, ashwaganda, magnesium, passionflower, skullcap, gamma aminobutryic acid, chamomile, and L-theanine St. John's wort (Hypericum perforatum) is a small, yellow-flowered herb that has been used for centuries for treating mental disorders and nerve pain. It is known in the art that St. John's wort is used for treating depression, having fewer side effects than prescription drugs and acting on the same pivotal neurotransmitter systems that mediate anxiety and depression several neurotransmitter systems in the brain that mediate anxiety and depression [15]. Through its effects as a serotonin reuptake inhibitor, this herb has been demonstrated to prevent neurons from reabsorbing serotonin, thereby increasing the availability of this neurotransmitter, which has positive effects on mood. St. John's wort is also a GABA agonist [16]. Indeed, animal studies have proven that St. John's wort affords changes in neurotransmitter concentrations in brain areas involved in depression, and that its mechanism of action of is comparable to that of standard anti-depressant drugs, fluoxetine (known as the tradenames Prozac and Sarafem among others) and imipramine (Tofranil) [17].

In another embodiment, gamma aminobutyic acid (GABA) is administered to a mammal along with said composition of ingredients comprising niacin, ashwaganda, magnesium, passionflower, skullcap, St. John's wort, chamomile, and L-theanine GABA is synthesized form the amino acid glutamate in the brain and can also be taken as a supplement. Neuronal activity in the brain is regulated by the balance between excitatory (via glutamate) and inhibitory signals (GABA). Accordingly, low levels of GABA are associated with restlessness, anxiety, and insomnia [18].

Clinical studies have demonstrated the efficacy of GABA supplementation for relief of stress and anxiety. A published article revealed increases in alpha brain waves (associated with relaxation) by electroencephalograms sixty minutes after oral administration of GABA to volunteers [19]. Benzodiazepines, such as Valium and Xanax®, stimulate GABA receptors, thus mimicking the calming effects of GABA but again do not fix the lack of GABA production [20]. Therefore, dietary supplementation with GABA offers a natural and effective alternative for treating these conditions.

In another embodiment, chamomile is administered to a mammal along with said composition of ingredients comprising niacin, ashwaganda, magnesium, passionflower, skullcap, St. John's wort, gamma aminobutyric acid, and L-theanine Chamomile (Matricaria recutita) is a flowering plant of the daisy family that has been used as a traditional medicine for thousands of years to calm anxiety. Favonoids in chamomile are anxiolytic through their effects in activating the GABA pathway in the brain, even enhancing the effects of diazepam (Valium) in in vitro experiments [21].

Clinical studies have demonstrated that ingestion chamomile has effects in alleviating anxiety and for treating syndromes associated with both anxiety and depression [22, 23]. Chamomile extract, prepared as 220 mg capsules (escalating weekly doses up to a maximum of 1, 100 mg/day) was shown to alleviate anxiety, according to Hamilton Anxiety Rating (HAM-A) scores in a randomized, placebo-controlled trial involving 61 patients afflicted with Generalized Anxiety Disorder [22]. In one embodiment of the present invention, chamomile is administered to a mammal at a concentration between 50-1000 mg along with a combination of ingredients comprising niacin, magnesium, ashwagandha, passion flower, skullcap, St. John's Wort, gamma aminobutryic acid, and L-theanine for treatment of anxiety and/or depression.

In another embodiment, L-theanine is administered to a mammal along with said composition of ingredients comprising niacin, ashwaganda, magnesium, passionflower, skullcap, St. John's wort, and gamma aminobutyric acid. L-theanine is an amino acid found in green tea that is known in the art to have a calming effect on moods. Theanine passes through the blood brain barrier where it inhibits glutamate (excitatory) pathways and stimulates GABA (inhibitory pathways) [24].

In the present invention, the methods and techniques for preparation of the composition of ingredients comprising niacin, ashwaganda, magnesium, passionflower, skullcap, St. John's wort, and gamma aminobutyric acid are known in the art. Where applicable, these practices involve harvesting herbs from the appropriate sources and preparing pharmaceutical-grade extracts, vitamins and minerals. In one embodiment of the present invention, these ingredients are administered orally in capsules; however, this combination of ingredients can be administered to a mammal, either individually or collectively, using other delivery vehicles or routes of administration.

The herb-based composition of the present invention can be used in beverages, tonics, infusions, or food stuffs alone, or in combination with other dietary supplements or therapeutics that are known in the art. The herb-based composition of the present invention can be used alone or further formulated with pharmaceutically acceptable compounds, vehicles, or adjuvants with a favorable delivery profile, i.e., suitable for delivery to a subject. A composition of the present invention may be formulated to be compatible with its intended route of administration; for example (but not limited to) oral compositions, which generally include an inert diluent or an edible carrier. Oral compositions can be enclosed in a variety of delivery vehicles known in the art, including gelatin capsules, tablets, reconstitutable powders, lozenges, liquids, suspensions, emulsions, capsules, and combinations thereof.

### REFERENCES

1. Nutt, D. J. and A. L. Malizia, New insights into the role of the GABA(A)-benzodiazepine receptor in psychiatric disorder. Br J Psychiatry, 2001. 179: p. 390-6.

2. Goldsmith, G. A., Niacin-tryptophan relationships in man and niacin requirement. Am J Clin Nutr, 1958. 6(5): p. 479-86.

3. Badawy, A. A., Pellagra and Alcoholism: A Biochemical Perspective. Alcohol Alcohol, 2014.

4. Tonge, W. L., Nicotinic acid in the treatment of depression. Ann Intern Med, 1953. 38(3): p. 551-3.

5. Bhattacharya, S. K., et al., Anxiolytic-antidepressant activity of Withania somnifera glycowithanolides: an experimental study. Phytomedicine, 2000. 7(6): p. 463-9.

6. Chandrasekhar, K., J. Kapoor, and S. Anishetty, A prospective, randomized double-blind, Placebo-controlled study of safety and efficacy of a high-concentration full-spectrum extract of ashwagandha root in reducing stress and anxiety in adults. Indian J Psychol Med, 2012. 34(3): p. 255-62.

7. Movafegh, A., et al., Preoperative oral Passiflora incarnata reduces anxiety in ambulatory surgery patients: a double-blind, placebo-controlled study. Anesth Analg, 2008. 106(6): p. 1728-32.

8. Akhondzadeh, S., et al., Passionflower in the treatment of opiates withdrawal: a double-blind randomized controlled trial. J Clin Pharm Ther, 2001. 26(5): p. 369-73.

9. Grundmann, O., et al., Anxiolytic activity of a phytochemically characterized Passiflora incarnata extract is mediated via the GABAergic system. Planta Med, 2008. 74(15): p. 1769-73.

10. Derom, M. L., et al., Magnesium and depression: a systematic review. Nutr Neurosci, 2013. 16(5): p. 191-206.

11. Eby, G. A. and K. L. Eby, Rapid recovery from major depression using magnesium treatment. Med Hypotheses, 2006. 67(2): p. 362-70.

12. Wolfson, P. and D. L. Hoffmann, An investigation into the efficacy of Scutellaria lateriflora in healthy volunteers. Altern Ther Health Med, 2003. 9(2): p. 74-8.

13. Lohani, M., et al., Anti-oxidative and DNA protecting effects of flavonoids-rich Scutellaria lateriflora. Nat Prod Commun, 2013. 8(10): p. 1415-8.

14. Awad, R., et al., Phytochemical and biological analysis of skullcap (Scutellaria lateriflora L.): a medicinal plant with anxiolytic properties. Phytomedicine, 2003. 10(8): p. 640-9.

15. Muller, W. E., Current St John's wort research from mode of action to clinical efficacy. Pharmacol Res, 2003.47(2): p. 101-9.

16. Butterweck, V., Mechanism of action of St John's wort in depression:what is known? CNS Drugs, 2003. 17(8): p. 539-62.

17. Bukhari, I. A. and A. Dar, Behavioral profile of Hypericum perforatum (St. John's Wort) extract. A comparison with standard antidepressants in animal models of depression. Eur Rev Med Pharmacol Sci, 2013. 17(8): p. 1082-9.

18. Nemeroff, C. B., The role of GABA in the pathophysiology and treatment of anxiety disorders. Psychopharmacol Bull, 2003. 37(4): p. 133-46.

19. Abdou, A. M., et al., Relaxation and immunity enhancement effects of gamma-aminobutyric acid (GABA)administration in humans. Biofactors, 2006. 26(3): p. 201-8.

20. Alramadhan, E., et al., Dietary and botanical anxiolytics. Med Sci Monit, 2012. 18(4): p. RA40-8.

21. Campbell, E. L., M. Chebib, and G. A. Johnston, The dietary flavonoids apigenin and (-)-epigallocatechin gallate enhance the positive modulation by diazepam of the activation by GABA of recombinant GABA(A)receptors. Biochem Pharmacol, 2004. 68(8): p. 1631-8.

22. Amsterdam, J. D., et al., A randomized, double-blind, placebo-controlled trial of oral Matricaria recutita (chamomile)extract therapy for generalized anxiety disorder. J Clin Psychopharmacol, 2009. 29(4): p. 378-82.

23. Amsterdam, J. D., et al., Chamomile (Matricaria recutita)may provide antidepressant activity in anxious, depressed humans: an exploratory study. Altern Ther Health Med, 2012. 18(5): p. 44-9.

24. Kakuda, T., Neuroprotective effects of theanine and its preventive effects on cognitive dysfunction. Pharmacol Res, 2011. 64(2): p. 162-8.

What is claimed is:

1. A nutraceutical formulation for the management and prevention of anxiety and depression comprising the following naturally occurring substances: niacin, magnesium, ashwagandha, passion flower, skullcap, St. John's Wort, gamma aminobutryic acid, chamomile, and L-theanine.

2. The nutraceutical formulation of claim 1, wherein said combination of naturally occurring substances comprises the following: niacin at a concentration between 10-1000 mg, niacin, magnesium at a concentration between 50-1000 mg, ashwagandha at a concentration between 100-500 mg, passion flower at a concentration between 100-1000 mg, skullcap at a concentration between 100-2000 mg, St. John's wort at a concentration between 100-1000 mg, gamma aminobutyric acid at a concentration between 100-1000 mg, chamomile at a concentration between 50-1000 mg, and L-theanine at a concentration between 50-500 mg.

3. The nutraceutical formulation of claim 1, wherein said combination of naturally occurring substances comprises the following: niacin at approximately 50 mg, magnesium at approximately 50 mg, ashwagandha at approximately 200 mg, passion flower at approximately 100 mg, skullcap at approximately 100 mg, St. John's wort at approximately 100 mg, gamma aminobutyric acid at approximately 100 mg, chamomile at approximately 50 mg, and L-theanine at approximately 50 mg.

4. The nutraceutical formulation of claim 3, wherein said combination of ingredients is incorporated into capsules suitable for oral consumption.

5. A method of treating anxiety and/or depression in a mammal in need, comprising identifying a mammal suffering from anxiety and/or depression and administering a combination of naturally occurring substances comprising: niacin, magnesium, ashwagandha, passion flower, skullcap, St. John's Wort, gamma aminobutryic acid, chamomile, and L-theanine.

6. The method of claim 5, wherein said combination of naturally occurring substances comprises the following: niacin at a concentration between 10-1000 mg, niacin, magnesium at a concentration between 50-1000 mg, ashwagandha at a concentration between 100-500 mg, passion flower at a concentration between 100-1000 mg, skullcap at a concentration between 100-2000 mg, St. John's wort at a concentration between 100-1000 mg, gamma aminobutyric acid at a concentration between 100-1000 mg, chamomile at a concentration between 50-2000 mg, and L-theanine at a concentration between 50-500 mg.

7. The method of claim 5, wherein said combination of naturally occurring substances is comprised of: niacin at approximately 50 mg, magnesium at approximately 50 mg, ashwagandha at approximately 200 mg, passion flower at approximately 100 mg, skullcap at approximately 100 mg, St. John's wort at approximately 100 mg, gamma aminobutyric acid at approximately 100 mg, chamomile at approximately 50 mg, and L-theanine at approximately 50 mg.

8. The method of claim 5, wherein said combination of ingredients is incorporated into capsules suitable for oral consumption.

9. The method of claim 5, wherein said combination of ingredients is administered orally.

10. The method of claim 5, wherein said combination of ingredients is administered twice daily to a mammal in need of said formulation.

11. The method of claim 5, wherein the anxiety and/or depression is selected from the group consisting of: a) panic disorder; b) obsessive compulsive disorder; c) post-traumatic stress disorder; d) social anxiety disorder; e) specific phobias; f) generalized anxiety disorder; g) depression; h) insomnia.

12. A method of treating neurological conditions in a mammal, comprising the steps of: identifying a mammal suffering from a neurological condition; administering a combination of ingredients comprising: niacin, magnesium, ashwagandha, passionflower, skullcap, St. John's Wort, gamma aminobutryic acid, chamomile, and L-theanine, and increasing the activity of one of the following neurotransmitter pathways: Serotonin, GABA.

13. The method of claim 12, wherein said increased activity of neurotransmitter pathways results in an effect selected from the group consisting of: amelioration of anxiety, amelioration of depression, improvement of sleep, improvement of memory, and reduction of stress-related behaviors.

14. The method of claim 12, wherein said combination of ingredients are administered at the following concentrations: niacin at a concentration between 10-1000 mg, niacin, magnesium at a concentration between 50-1000 mg, ashwagandha at a concentration between 100-500 mg, passion flower at a concentration between 100-1000 mg, skullcap at a concentration between 100-2000 mg, St. John's wort at a concentration between 100-1000 mg, gamma aminobutyric acid at a concentration between 100-1000 mg, chamomile at a concentration between 50-2000 mg, and L-theanine at a concentration between 50-500 mg.

15. The method of claim 12, wherein said combination of ingredients is comprised of: niacin at approximately 50 mg, magnesium at approximately 50 mg, ashwagandha at approximately 200 mg, passion flower at approximately 100 mg, skullcap at approximately 100 mg, St. John's wort at approximately 100 mg, gamma aminobutyric acid at approximately 100 mg, chamomile at approximately 50 mg, and L-theanine at approximately 50 mg.
